(19) United States
(12) Patent Application Publication
(10) Pub. No.: US 2022/0341946 A1
(43) Pub. Date: Oct. 27, 2022

(54) BIOMARKERS FOR ASSESSING EXPLANT ORGAN VIABILITY

(71) Applicant: University of Newcastle Upon Tyne, Newcastle upon Tyne, Tyne and Wear (GB)

(72) Inventors: Colin WILSON, Newcastle upon Tyne (GB); Rodrigo FIGUEIREDO, Newcastle upon Tyne (GB); Matt WRIGHT, Newcastle upon Tyne (GB); Alistair LEITCH, Newcastle upon Tyne (GB)

(21) Appl. No.: 17/754,313

(22) PCT Filed: Oct. 1, 2020
(86) PCT No.: PCT/GB2020/052397

§ 371 Date: Mar. 30, 2022

(30) Foreign Application Priority Data
Oct. 4, 2019 (GB) 1914399.9

(51) Int. Cl. G01N 33/68; A01N 1/02

(52) U.S. Cl. CPC G01N 33/6893; A01N 1/0226; G01N 2333/4737; G01N 2333/475; G01N 2333/5409; G01N 2333/5434; G01N 2333/5443; G01N 2333/5446

(57) ABSTRACT

Aspects of the present invention relate to the assessment of explant organ viability prior to transplantation. Particularly, although not exclusively, aspects of the present invention relate to biomarkers which can be used to inform a decision as to whether an organ is suitable for transplantation into a recipient. In certain embodiments, the organ is undergoing hypothermic perfusion following retrieval from a donor.

### FIELD OF INVENTION

Aspects of the present invention relate to the assessment of explant organ viability prior to transplantation. Particularly, although not exclusively, aspects of the present invention relate to biomarkers which can be used to inform a decision as to whether an organ is suitable for transplantation into a recipient. In certain embodiments, the organ is undergoing hypothermic perfusion following retrieval from a donor.

### BACKGROUND TO THE INVENTION

Transplantation has transformed the lives of millions and is cost-effective when compared with kidney dialysis and chronic liver disease management. Liver transplantation can be a highly successful treatment for end-stage liver disease, fulminant hepatic failure and early-stage primary liver cancer. Improvements in outcomes from liver transplantation over the years have transformed it from an experimental procedure, to almost routine. The average 5-year survival post liver transplantation is now 80%. These improvements have led to an exponential growth in patients being added to the waiting list for liver transplantation, but organ donation has been unable to keep up with the rising demand. As a result, the waiting list for liver transplantation in the UK has risen significantly over the last 2 decades, with a significant mortality rate whilst on the waiting list, which is similar to other regions. The waiting list peaked in 2015/16, and there has been a decline since then from 611 to 359 patients 2018 (United Kingdom).

Donation after circulatory death (DCD) liver grafts are increasingly used for transplantation in an attempt to overcome the discrepancy between the number of available donors and the number of patients waiting for a liver transplant. DCD donors go through an inevitable period of warm ischaemia between the withdrawal of life support and circulatory arrest. This first period of warm ischaemia and the following cold ischaemia during transportation lead to depletion of intracellular energy sources, such as ATP, cellular injury and dysfunction. Consequently, DCD liver grafts are associated with increased reperfusion injury, post-transplantation morbidity and graft loss. Increased use of DCD livers without changes to preservation methods would most likely result in inferior outcomes to those transplanted. There is evidence to demonstrate the inferiority of DCD livers when compared with livers from donors after brain death (DBD). DCD livers have higher rates of early allograft dysfunction (EAD), biliary complications, acute kidney injury and worse survival compared with DBD livers.

Although some centres have demonstrated that with very judicious donor and recipient selection, adequate outcomes can be achieved; there is a growing perception that static cold storage (SCS) in an ice box is inadequate preservation for these sub-optimal organs. For sustained widespread safe utilisation of DCD livers, novel methods of organ preservation need to be developed.

Currently far more livers are retrieved from organ donors than are actually transplanted. In the UK in 2018, around 1500 livers were removed for transplant, but only 1000 were actually transplanted. The bile ducts are particularly prone to damage in DCD donors and up to 40% of recipients need a re-transplant after a DCD transplant or die. Worldwide thousands of donated organs are not used because of concerns about how they will function after transplant. The imbalance between the number of organs available and the demand has led many researchers to look for novel ways to rescue organs previously considered unsuitable for transplantation.

SCS has been an excellent preservation modality for many years but with the increasing numbers of elderly, overweight and DCD donors, the incidence of early allograft dysfunction and primary non-function has been increasing dramatically. Machine perfusion is recognised as being one of the most significant advances in the field of organ transplantation over the last twenty years. For livers, there are two main types of perfusion approaches used in the clinic. One is perfusion with blood or alternative oxygen carriers at physiologic normothermic or subnormothermic conditions. Normothermic machine perfusion (NMP) is used to minimize the duration of cold storage of the liver either in situ, i.e. in the donor before procurement or ex situ during or after organ transport to the transplant site. The second perfusion technique is hypothermic machine perfusion (HOPE), a perfusion technique developed in the United States by Guaerra and in Europe by Dutowoski (Zurich) and Porte (Gronigen) in the early 2000's. In 2013 Newcastle NIHR Blood Transplant Research Unit performed the first procedure in the UK. This technique has also been used to perfuse kidneys for transplantation since 1960.

HOPE aims to recondition livers prior to implementation, to reduce some of the deleterious effects of static cold storage, and subsequently reduce ischaemic reperfusion injury. The liver is retrieved and transported as standard to the transplanting centre when it is prepared for transplantation and placed on perfusion whilst the recipient is being anaesthetised. The technique means that livers which previously could not be used for transplant become viable. At present the decision on whether to accept a particular liver for transplant is complex and requires the transplanting surgeon to weigh up multiple factors from both the donor and recipient. The British Transplantation Society have published guidelines on the use of donor organs and use criteria such as age of donor, duration of cold ischemic time, level of steatosis and length of stay in intensive care.

There is a relatively high proportion of organs currently retrieved that do not go on to be transplanted. The principal reason for discard of a liver that has been retrieved with the intention of being transplanted is fear that the liver will not function adequately after transplantation, usually in the setting of steatosis, prolonged warm ischemia, adverse hemodynamic characteristics during the DCD withdrawal phase, or prolonged cold ischemia. Although the liver may have functioned well in the donor, warm and cold ischaemia impose an unpredictable injury on the liver that may manifest only after reperfusion in the recipient. The ability to objectively predict future function in the recipient is the ultimate aim of real-time viability assessment.

It is an aim of certain embodiments to at least partially overcome the technical problems associated with the prior art.

It is an aim of certain embodiments of the present invention to provide a method of assessing the viability of an organ undergoing perfusion.

It is an aim of certain embodiments to provide a method of transplanting an organ that has been assessed and deemed suitable for transplantation.

### SUMMARY OF THE INVENTION

In a broad aspect of the present invention, there is provided a method of assessing suitability of an explant organ for transplantation into a subject in need thereof, the method comprising:
a) obtaining a sample of a perfusate fluid in which an explant organ is located;
b) determining the amount in the sample of at least one protein selected from:
   i) IL-5;
   ii) IL-12;
   iii) IL-15;
   iv) IL-16;
   v) C-reactive protein (CRP); and
   vi) VEGF; and
c) determining whether the explant organ is suitable for transplantation based on whether the amount of the at least one protein in the sample is greater or less than a predetermined value.

Aptly, the VEGF protein is VEGF-A.

In certain embodiments, the method comprises determining that the organ is suitable for transplantation if the amount of at least one protein is less than the predetermined value.

In a further aspect of the present invention, there is provided a method of assessing suitability of an explant organ for transplantation into a subject in need thereof, the method comprising:
a) obtaining a sample of a perfusate fluid in which an explant organ is located;
b) determining an amount in the sample of at least three proteins selected from:
   i) IL-5;
   ii) IL-12;
   iii) IL-15;
   iv) IL-16;
   v) C-reactive protein (CRP); and
   vi) VEGF; and
c) determining whether the explant organ is suitable for transplantation based on whether a respective amount of each protein in the sample is greater or less than a respective predetermined value.

In certain embodiments, the method comprises determining that the explant organ is suitable for transplantation if the respective amount of each of the at least three proteins is less than the respective predetermined value.

In certain embodiments, the step of determining an amount in the sample comprises quantifying the amount present in the sample of at least three proteins selected from:
i) IL-5;
ii) IL-12;
iii) IL-15;
iv) IL-16;
v) CRP; and
vi) VEGF; and
   wherein the step of determining whether the explant organ is suitable for transplantation comprises;
a) for each protein, determining whether the amount of the protein is greater or less than a predetermined value;
b) assigning a score of 1 to the protein if the amount is greater than the predetermined value; and
c) adding together the score of each protein; wherein the explant organ is determined as suitable for transplantation if the additive score of the proteins is less than 3.

In certain embodiments, the step of determining an amount in the sample comprises quantifying the amount present in the sample of at least four proteins selected from:
i) IL-5;
ii) IL-12;
iii) IL-15;
iv) IL-16;
v) CRP; and
vi) VEGF, and
   wherein the step of determining whether the explant organ is suitable for transplantation comprises;
a) for each protein, determining whether the amount of the protein is greater or less than a predetermined value;
b) assigning a score of 1 to the protein if the amount is greater than the predetermined value and optionally assigning a score of 0 to the protein if the amount is less than or equal to the predetermined value; and
c) adding together the score of each protein; wherein the explant organ is determined as suitable for transplantation if the additive score of the proteins is less than 3.

In certain embodiments, wherein the step of determining an amount comprises quantifying the amount present in the sample of at least five proteins selected from:
i) IL-5;
ii) IL-12;
iii) IL-15;
iv) IL-16;
v) CRP; and
vi) VEGF, and
   wherein the step of determining whether the explant organ is suitable for transplantation comprises;
a) for each protein, determining whether the amount of the protein is greater or less than a predetermined value;
b) assigning a score of 1 to the protein if the amount is greater than the predetermined value and optionally assigning a score of 0 to the protein if the amount is less than or equal to the predetermined value; and
c) adding together the score of each protein; wherein the explant organ is determined as suitable for transplantation if the additive score of the proteins is less than 3.

In certain embodiments, the method comprises quantifying the amount present in the sample of IL-5, IL-12, IL-15, IL-16, CRP and VEGF, and further comprises:
a) assigning a score of 1 to the protein if the amount is greater than the predetermined value and optionally assigning a score of 0 to the protein if the amount is less than or equal to the predetermined value; and
b) adding together the score of each protein; wherein the explant organ is determined as suitable for transplantation if the additive score of the proteins is less than 3.

In certain embodiments, the predetermined value(s) are selected from one or more of the group consisting of:
a) between about 0.62 to about 0.63 pg/ml of IL-5 e.g. about 0.6221 pg/ml;
b) between about 14.4 to about 14.5 pg/ml of IL-12 e.g. about 14.46 pg/ml;
c) between about 2.19 to about 2.2 pg/ml of IL-15 e.g. about 2.192 pg/ml;
d) between about 1235 to about 1250 pg/ml of IL-16 e.g. about 1244 pg/ml;
e) between about 6.28 to about 6.29 pg/ml of VEGF e.g. about 6.285 pg/ml; and
e) between about 2.39 to about 2.4 pg/ml CRP e.g. about 2.394 pg/ml.

In certain embodiments, the method comprises quantifying the amount present in the sample of at least two proteins selected from:
i) IL-5;
ii) IL-12;
iii) IL-15;
iv) IL-16;
v) CRP; and
vi) VEGF,
   wherein the step of determining whether the explant organ is suitable for transplantation comprises;
a) determining whether the amount of at least one protein is greater or less than a predetermined value;
b) assigning a score of 1 to the protein if the amount is greater than the predetermined value and optionally assigning a score of 0 to the protein if the amount is less than or equal to the predetermined value; and
c) adding together the score of each protein; wherein the explant organ is determined as suitable for transplantation if the additive score of the proteins is less than 3.

In certain embodiments, the method comprises quantifying the amount present in the sample of at least three proteins selected from:
i) IL-5;
ii) IL-12;
iii) IL-15;
iv) IL-16;
v) CRP; and
vi) VEGF;
   wherein the step of determining whether the explant organ is suitable for transplantation comprises;
a) determining whether the amount of the at least one protein is greater or less than a predetermined value;
b) assigning a score of 1 to the protein if the amount is greater than the predetermined value and optionally assigning a score of 0 to the protein if the amount is less than or equal to the predetermined value; and
c) adding together the score of each protein; wherein the explant organ is determined as suitable for transplantation if the additive score of the proteins is less than 3.

In certain embodiments, the method comprises quantifying the amount present in the sample of at least four proteins selected from:
i) IL-5;
ii) IL-12;
iii) IL-15;
iv) IL-16;
v) CRP; and
vi) VEGF,
   wherein the step of determining whether the explant organ is suitable for transplantation comprises;
a) determining whether the amount of the at least one protein is greater or less than a predetermined value;
b) assigning a score of 1 to the protein if the amount is greater than the predetermined value and optionally assigning a score of 0 to the protein if the amount is less than or equal to the predetermined value; and
c) adding together the score of each protein; wherein the explant organ is determined as suitable for transplantation if the additive score of the proteins is less than 3.

In certain embodiments, the method comprises quantifying the amount present in the sample of at least five proteins selected from:
i) IL-5;
ii) IL-12;
iii) IL-15;
iv) IL-16;
v) CRP; and
vi) VEGF,
   wherein the step of determining whether the explant organ is suitable for transplantation comprises;
a) determining whether the amount of the at least one protein is greater or less than a predetermined value;
b) assigning a score of 1 to the protein if the amount is greater than the predetermined value and optionally assigning a score of 0 to the protein if the amount is less than or equal to the predetermined value; and
c) adding together the score of each protein; wherein the explant organ is determined as suitable for transplantation if the additive score of the proteins is less than 3.

In certain embodiments, the method comprises quantifying the amount present in the sample of IL-5, IL-12, IL-15, IL-16, CRP and VEGF, and further comprises:
a) assigning a score of 1 to the protein if the amount is greater than the predetermined value and optionally assigning a score of 0 to the protein if the amount is less than or equal to the predetermined value; and
b) adding together the score of each protein; wherein the explant organ is determined as suitable for transplantation if the additive score of the proteins is less than 3.

In certain embodiments, the predetermined value for the amount of IL-5 in the sample is between about 0.62 pg/ml and about 0.63 pg/ml, for example about 0.6221 pg/ml. In certain embodiments, the predetermined value for the amount of IL-12 in the sample is between about 14.4 pg/ml and 14.5 pg/ml. For example, the predetermined value for the amount of IL-12 is about 14.46 pg/ml.

In certain embodiments, the predetermined value for the amount of IL-15 in the sample is between about 2.19 pg/ml to about 2.20pg/ml. For example, the predetermined value for the amount of IL-15 may be about 2.192 pg/ml. In certain embodiments, the predetermined value for the amount of IL-16 is between about 1235 pg/ml and about 1250 pg/ml. For example, the predetermined value for the amount of IL-16 is about 1244 pg/ml.

In certain embodiments, the predetermined value for the amount of VEGF is between about 6.28 pg/ml and about 6.29 pg/ml. For example, the predetermined value of VEGF is about 6.285 pg/ml. Aptly, the VEGF protein is VEGF-A.

In certain embodiments, the predetermined value for the amount of CRP is between about 2.39 pg/ml and about 2.40 pg/ml. For example, the predetermined value for the amount of CRP may be about 2.394 pg/ml.

In certain embodiments, the explant organ is undergoing hypothermic perfusion.

In certain embodiments, the method further comprises, prior to step (a), locating the explant organ in the perfusate fluid.

In certain embodiments, the method comprises inserting an organ into a perfusion machine comprising the perfusate fluid. Aptly, the perfusate fluid is approximately 5 -10° C. e.g. about 8° C. when it enters the explant organ. In certain embodiments, the explant organ is undergoing hypothermic perfusion. In certain embodiments, the sample is obtained approximately 15-30 minutes subsequent to the initiation of the perfusion. For example, the sample may be obtained 15 minutes, 20 minutes, 25 minutes or 30 minutes after the start of perfusion of the organ.

In certain embodiments, the method comprises, if the amount of more than one protein is determined, determining the amounts of proteins simultaneously.

In certain embodiments, the perfusate fluid comprises one or more of the following components:
i) pentafraction,
ii) lactobionic acid
iii) potassium phosphate monobasic
iv) magnesium sulfate heptahydrate
v) raffinose pentahydrate;
vi) adenosine;
vii) allopurinol;
viii) glutathione;
ix) potassium hydroxide; and
x) sodium hydroxide.

In certain embodiments, the perfusate fluid further comprises N-acetylcysteine, dexamethasone and benzylpenicillin.

In certain embodiments, the organ is a liver, e.g. a human liver.

In certain embodiments, the method further comprises;
determining the amount in a further sample of at least one protein selected from:
a) IL-7;
b) IL-17a;
c) IL-16; and
d) CRP,
   wherein the further sample is obtained between about 80 minutes and 150 minutes after the initiation of perfusion and further wherein the method comprises;
i) determining whether the amount of the at least one protein is greater or less than a predetermined value;
ii) assigning a score of 1 to the protein if the amount is greater than the predetermined value and optionally assigning a score of 0 to the protein if the amount is less than or equal to the predetermined value; and
iii) adding together the score of each protein; wherein the explant organ is determined as suitable for transplantation if the additive score of the proteins is less than 3.

In certain embodiments, the further sample is obtained at about 80, 85, 90, 95, 100, 105, 110, 115, 120, 125, 130, 135, 140, 145, 150, 155, 160, 165, 170, 175 and 180 minutes.

In certain embodiments, the predetermined value of IL-7 is between about 4.70 pg/ml and about 4.750 pg/ml e.g. about 4.717 pg/ml.

In certain embodiments, the predetermined value of IL-16 when sampled between about 80 to 120 minutes after the start of perfusion is between about 925 pg/ml to about 940 pg/ml e.g. about 934.3 pg/ml. In certain embodiments, the predetermined value of IL-17A is between about 3.60 pg/ml and about 3.70 pg/ml e.g. about 3.646 pg/ml. In certain embodiments, the predetermined value of CRP when determined in a sample taken between about 80 minutes and 120 minutes after the start of perfusion is between about 4.20 pg/ml and 4.30 pg/ml e.g. about 4.230 pg/ml.

In certain embodiments, the method comprises, if the amount of more than one protein is determined, determining the amounts of proteins simultaneously.

In certain embodiments, the step of detecting the amount of the at least one protein comprises performing an immunoassay, wherein optionally the immunoassay is selected from an ELISA, a radioimmunoassay, automated immunoassay, cytometric based assay and immunoprecipitation assay.

In certain embodiments, the method further comprises contacting the sample with at least one detection molecule, wherein the detection molecule is specific to a protein selected from:
i) IL-5;
ii) IL-12;
iii) IL-15;
iv) IL-16;
v) C-reactive protein (CRP); and
vi) VEGF.

In certain embodiments, the method comprises contacting the sample with a plurality of detection molecules, the plurality of detection molecules comprising a detection molecule specific to a protein selected from:
i) IL-5;
ii) IL-12;
iii) IL-15;
iv) IL-16;
v) C-reactive protein (CRP); and
vi) VEGF.

In certain embodiments, the plurality of detection molecules comprises a detection molecule specific to IL-5. Alternatively or in addition, the plurality of detection molecules comprises a detection molecule specific to IL-12. Alternatively or in addition, the plurality of detection molecules comprises a detection molecule specific to IL-15. Alternatively or in addition, the plurality of detection molecules comprises a detection molecule specific to IL-16. Alternatively or in addition, the plurality of detection molecules comprises a detection molecule specific to C-reactive protein (CRP). Alternatively or in addition, the plurality of detection molecules comprises a detection molecule specific to VEGF.

Alternatively or in addition, the plurality of detection molecules comprises a detection molecule specific to IL-7. Alternatively or in addition, the plurality of detection molecules comprises a detection molecule specific to IL-17a. Alternatively or in addition, the plurality of detection molecules comprises a detection molecule specific to IL-16. Alternatively or in addition, the plurality of detection molecules comprises a detection molecule specific to CRP.

In certain embodiments, the detection molecule comprises an antibody or fragment thereof. In certain embodiments, the detection molecule is an aptamer.

In certain embodiments, the method is an ex vivo method. In certain embodiments, the organ is obtained from a deceased donor, for example a donor after circulatory death.

In a further aspect of the present invention, there is provided a method of transplanting an organ into a subject recipient comprising:
a) performing the method of assessing suitability of an explant organ for transplantation as described herein;
b) transplanting the organ into the subject recipient if the organ is assigned a score of 3 or less; or discarding the organ if the organ is assigned a score of 4 or greater.

In certain embodiments, the organ is a liver. Aptly, the organ is a human liver.

In certain embodiments, the method further comprises obtaining the organ from a deceased donor. In certain embodiments, the donor has undergone circulatory death prior to donation.

In certain embodiments the method further comprises placing the organ in a hypothermic perfusion system. In certain embodiments, the subject is a human.

In a further aspect of the present invention, there is provided an assay device for use in determining whether an organ is suitable for transplantation into a subject, the assay comprising:
a) at least one reaction chamber; and
b) a plurality of detection molecules, the plurality comprising detection molecules that are specific to a protein selected from:
   i) IL-5;
   ii) IL-12;
   iii) IL-15;
   iv) IL-16;
   v) C-reactive protein (CRP); or
   vi) VEGF.

In certain embodiments, the assay device further comprises a plurality of detection molecules, the plurality comprising detection molecules which are specific to:
a) IL-7; or
b) IL-17a.

In certain embodiments, the detection molecules are antibodies or fragments thereof. In certain embodiments, the detection molecules are labelled.

In some embodiments, the method comprises contacting the sample or a portion thereof with a capture antibody and a detection antibody specific to one of (i) to (vi) and detecting the detection antibody.

In some embodiments, the method comprises contacting the sample or portion thereof with a plurality of capture antibodies and a plurality of detection antibodies, each one of the detection antibodies specifically binding to one of the proteins listed in (i) to (iv).

In certain embodiments, the method comprises simultaneous or substantially simultaneous detection and optionally quantification of two or more of the proteins listed under (i) to (vi).

Certain embodiments of the present invention provide the benefit of providing an opportunity for real-time assessment of graft function and viability prior to implantation; thereby providing a powerful tool for predicting the risk to the recipient from receiving a particular liver.

Certain embodiments of the present invention relate to the use of organs such as livers which are undergoing Hypothermic Oxygenated Perfusion (HOPE).

According to certain embodiments, the perfusion period allows for viability testing and other forms of organ assessment, and perfusion parameters to have a predictive value for organ outcome.

Certain embodiments are based on the identification of a molecular signature of the perfusion blend associated with transplantation success (Interleukin 5, IL-12, IL-15, IL-16, CRP and VEGF). Further details are provided herein below.

### BRIEF DESCRIPTION OF THE FIGURES

Certain embodiments of the invention will be described in more detail below with reference to the accompanying figures in which:

FIG. 1 includes graphs which illustrate the concentration of various proteins in a perfusate sample over time of perfusion. The red line (squares) illustrate the concentration in a liver that has been discarded as being considered unsuitable for transplantation. The blue line (circles) illustrate the concentration of various proteins in a perfusate obtained from a liver assessed as being suitable for transplantation;

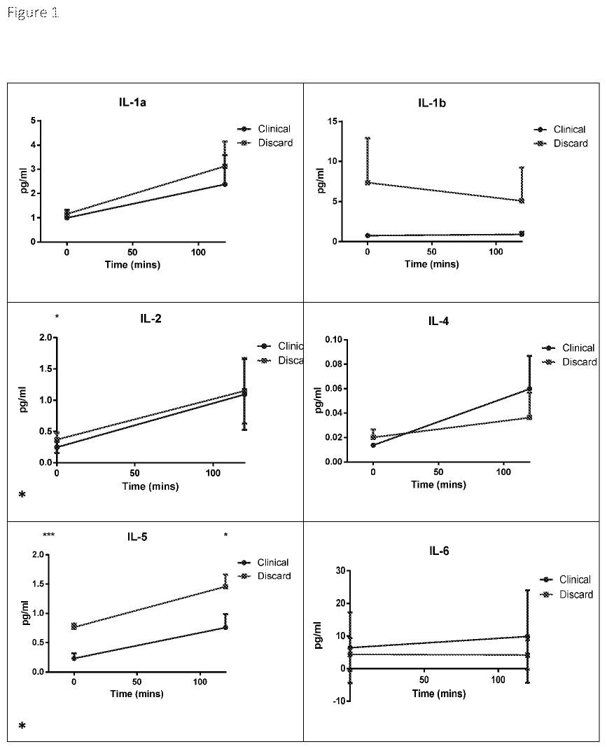

FIG. 2 illustrates a total score of 10 proteins in a perfusate fluid in which is DCD liver is placed;

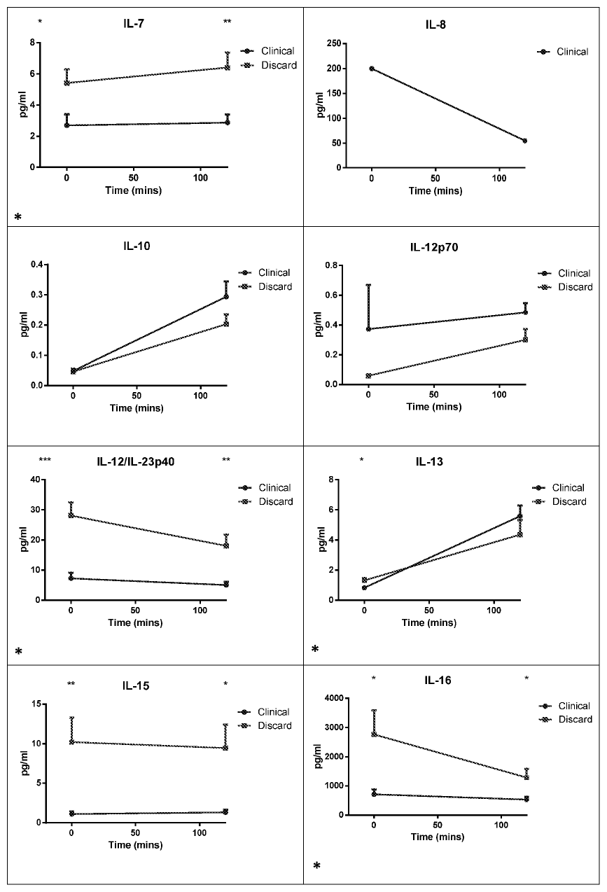

FIG. 3 shows the amino acid sequence of human IL-5 (SEQ. ID. No 1);

FIG. 4 shows the amino acid sequence of human IL-12 (SEQ. ID. No 2);

FIG. 5 shows the amino acid sequence of human IL-16 (SEQ. ID. No 3);

FIG. 6 shows the amino acid sequence of human IL-15 (SEQ. ID. No 4);

FIG. 7 shows the amino acid sequence of human CRP (SEQ. ID. No 5);

FIG. 8 shows the amino acid sequence of an isoform of human VEGF-A (SEQ. ID. No 6);

FIG. 9 shows the amino acid sequence of human IL-7 (SEQ. ID. No 7); and

FIG. 10 shows the amino acid sequence of a pro form of human IL-17a (SEQ. ID. No 8).

### DETAILED DESCRIPTION OF CERTAIN EMBODIMENTS

The practice of embodiments of the present invention employs, unless otherwise indicated, conventional techniques of chemistry, molecular biology, pharmaceutical formulation, pharmacology and medicine, which are within the skill of those working in the art.

Most general chemistry techniques can be found in Comprehensive Heterocyclic Chemistry IF (Katritzky et al., 1996, published by Pergamon Press); Comprehensive Organic Functional Group Transformations (Katritzky et al., 1995, published by Pergamon Press); Comprehensive Organic Synthesis (Trost et al. 1991, published by Pergamon); Heterocyclic Chemistry (Joule et al. published by Chapman & Hall); Protective Groups in Organic Synthesis (Greene et al., 1999, published by Wiley-Interscience); and Protecting Groups (Kocienski et al., 1994).

Most general molecular biology techniques can be found in Sambrook et al, Molecular Cloning, A Laboratory Manual (2001) Cold Harbor-Laboratory Press, Cold Spring Harbor, N.Y. or Ausubel et al., Current Protocols in Molecular Biology (1990) published by John Wiley and Sons, N.Y.

Most general pharmaceutical formulation techniques can be found in Pharmaceutical Preformulation and Formulation (2^{nd} Edition edited by Mark Gibson) and Pharmaceutical Excipients: Properties, Functionality and Applications in Research and Industry (edited by Otilia M Y Koo, published by Wiley).

Most general pharmacological techniques can be found in A Textbook of Clinical Pharmacology and Therapeutics (5^{th} Edition published by Arnold Hodder).

Most general techniques on the prescribing, dispensing and administering of medicines can be found in the British National Formulary 72 (published jointly by BMJ Publishing Group Ltd and Royal Pharmaceutical Society).

Unless defined otherwise, all technical and scientific terms used herein have the same meaning as commonly understood by one of ordinary skill in the art to which this disclosure belongs. For example, the Concise Dictionary of Biomedicine and Molecular Biology, Juo, Pei-Show, 2^{nd} ed., 2002, CRC Press; The Dictionary of Cell and Molecular Biology, 3^{rd} ed., Academic Press; and the Oxford University Press, provide a person skilled in the art with a general dictionary of many of the terms used in this disclosure. For chemical terms, the skilled person may refer to the International Union of Pure and Applied Chemistry (IUPAC).

Units, prefixes and symbols are denoted in their Systeme International d′Unités (SI) accepted form. Numeric ranges are inclusive of the numbers defining the range.

In a broad aspect of the present invention, there is provided methods and assays for determining the viability of an ex-vivo organ for transplantation into a living subject.

As used herein, the term “organ” includes for example liver, kidney, heart, lung, pancreas, small intestine, and limb (such as arm or leg, or portion thereof), or extremity (such as hand, foot, finger, toe, or a portion thereof). As used herein, “organ” also includes other tissues, such as tissue grafts, such as composite tissue allografts. In certain embodiments, the organ is a liver. In certain embodiments the organ is a liver donated after death of a donor. In certain embodiments, the liver is donated after circulatory death of a donor.

In certain embodiments, the organ is undergoing perfusion. The term “perfusion” is used to describe the circulation of a fluid (also referred to as a perfusion solution or perfusate fluid) through an organ to supply the needs of the organ to retain its viability (for example, in an ex vivo system). In some examples, the perfusion solution includes an oxygen carrier (for example, a haemoglobin-based oxygen carrier) or an artificial haemoglobin alternative.

Machine perfusion refers to the introduction and removal of a perfusion solution to an organ by a mechanical device. Such devices may include one or more chambers for holding an organ and a perfusion solution or perfusate fluid, one or more pumps for delivery of the perfusion solution to the organ, one or more means to regulate temperature of the perfusion solution, and one or more means to oxygenate the perfusate fluid. In some examples, machine perfusion includes introduction of an oxygen carrying fluid into an organ and removal of oxygen depleted fluid from the organ by circulation of the oxygen carrying fluid through the organ.

Aptly, the method comprises obtaining a sample from a perfusate fluid. The perfusate fluid is aptly used to perfuse the organ. In certain embodiments, the perfusate fluid is from a hypothermic perfusion system. Hypothermic perfusion (HOPE) aims to recondition livers prior to implantation, to reverse some of the deleterious effects of Static Cold Storage (SCS), and subsequently reduce IRI (Ischemic Reperfusion Injury). In HOPE, the liver is retrieved and transported as standard to the transplanting centre, where it is prepared for transplantation (‘back-benched’) and placed on perfusion whilst the recipient is being anaesthetised and the explant hepatectomy is being performed. There are multiple proposed beneficial effects of performing HOPE. Firstly, there is a physical washout benefit that helps clear microcirculation in the liver, including diluting waste products and blood remnants. Secondly there is the provision of oxygen to sustain intracellular energy production.

Furthermore, there is evidence to demonstrate that HOPE increases ATP content more than 15-fold, which remains elevated after reperfusion (in discarded organs). There also appears to be a reduction in expression of pro-inflammatory cytokines, downregulation of Kupffer cell activity, and reducing vascular resistance. Oxygenation at low temperatures also has the potential benefit of not generating Reactive Oxygen Species (ROS), which has been implicated as a deleterious effect of normothermic perfusion.

There is agreement that the highest risk livers stand to benefit most from HOPE, but there is no consensus on selection criteria. Higher average donor risk in Europe compared with North

America means a variable definition of ‘extended criteria’. Schlegel et al. recently postulated an inclusion criteria for HOPE including DBD grafts with donor age >80 years, cold ischaemia >10 hours or macrosteatosis >20%. They also included DCD grafts with donor age >60 years, functional warm ischaemia >20 mins, cold ischaemia >6 hours, or macrosteatosis >5%.

HOPE may have advantages over SCS and NMP such as retrievals and organ transportation continue as normal, and all interventions occur at the transplanting centre, where equipment and expertise already exist, therefore implementation is logistically fairly uncomplicated. Hypothermia has the added advantage of being a lower risk option than normothermic perfusion, such that if perfusion were to fail for technical reasons, the liver is still kept cool, and therefore no worse than SCS.

In certain embodiments, the method comprises perfusing the organ including a non-pulsatile arterial perfusion. In certain embodiments, the method comprises arterially perfusing the organ at an oxygen concentration of between about 20 — 30 kPa. In certain embodiments, the temperature of the perfusate fluid is at around 8-10° C. when it enters the organ.

In certain embodiments, the method comprises perfusing the organ under the following conditions:

| | Conditions |
|---|---|
| Type of Perfusion | D-HOPE |
| Arterial Perfusion | Non-pulsatile perfusion |
| IVC | Cannulated |
| Duration of Perfusion | 80-150 mins |
| Oxygenation | 20-30 kPa |
| Device | Medtronic |
| Temperature | 8-10° C. |
| Flow | Variable on pressure |
| Pressure | 3 mmHg (PV) |
| | 25 mmHg (HA) |
| Perfusate | Belzer UW |
| Additives | Antioxidant and vasodilator |
| Perfusate volume | 3 L |

Biomarkers in Perfusion Fluid

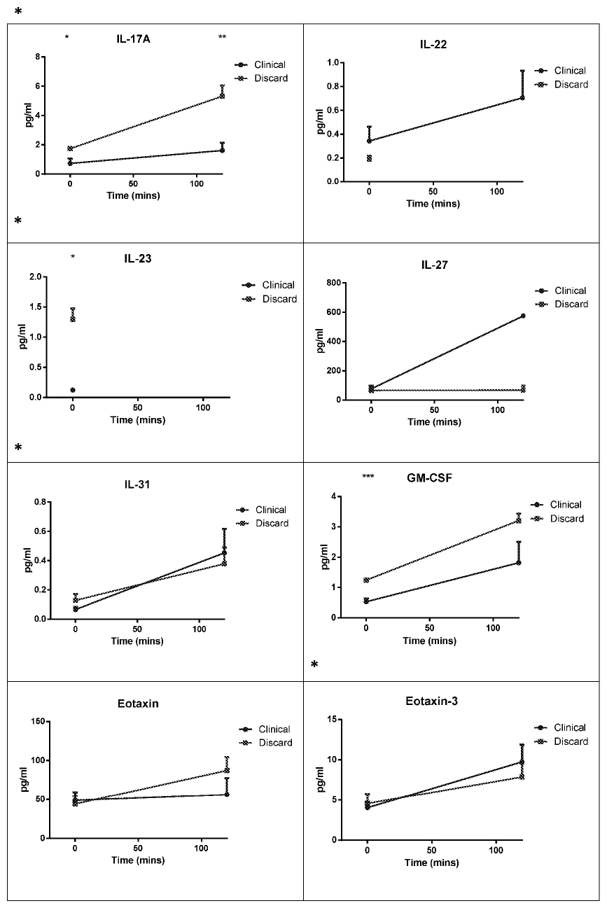

In certain embodiments, the method comprises detecting the concentration of IL-5 (e.g. human IL-5) in a perfusate fluid. IL-5 is an interleukin produced by Th2 helper cells and mast cells. It is a growth and differentiation factor for both B cells and eosinophils. The amino acid sequence of human IL-5 is disclosed under accession number UnitProtKB-P05113v1. The sequence is shown in FIG. 3 (SEQ. ID. No. 1)

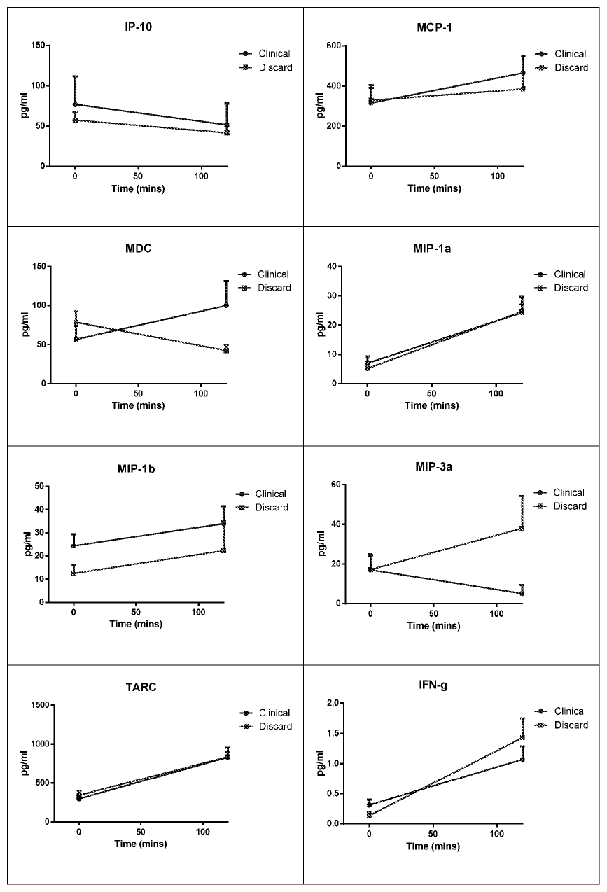

In certain embodiments, the method comprises detecting the concentration of IL-12 (e.g. human IL-12) in a perfusate fluid. IL-12 is an interleukin that is produced by dendritic cells, macrophages, neutrophils and human B-lymphoblastoid cells. IL-12 promotes development of Th1 responses and induces IFNγ production by T cells and NK cells. The amino acid sequence of human IL-12 is disclosed under accession number UniProtKB-P29459 version 2 (IL12A_HUMAN). The sequence is shown in FIG. 4 (SEQ. ID. No. 2)

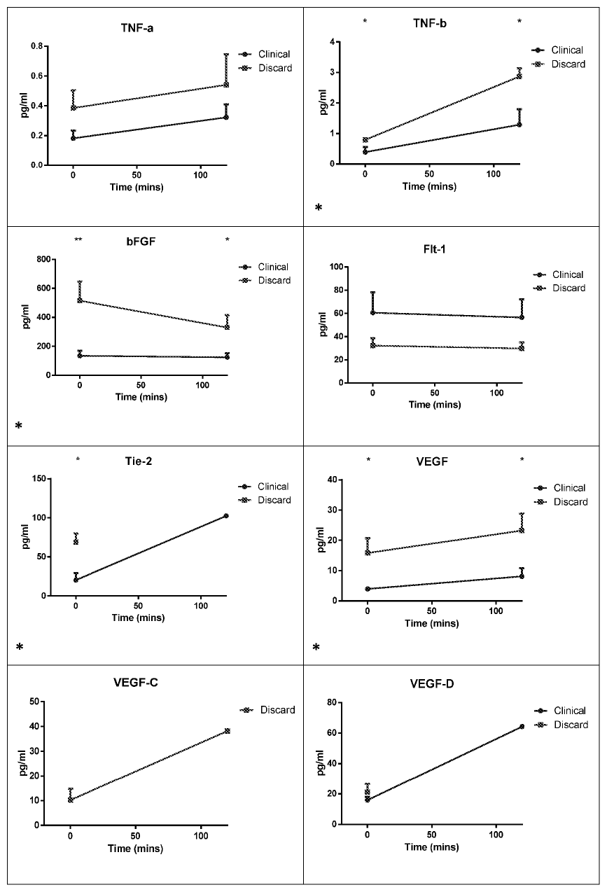

In certain embodiments, the method comprises detecting the concentration of IL-16 (e.g. human IL-16) in a perfusate fluid. IL-16 is a cytokine released by a variety of cells including lymphocytes and acts as a chemoattractant for immune cells expressing CD4. There are a number of isoforms of which the canonical amino acid sequence is disclosed under accession number UniProt Q14005-1. The amino acid sequence of human pro-IL16 is shown in FIG. 5 (SEQ. ID. No. 3). Pro-IL16 is subsequently cleaved to form IL-16.

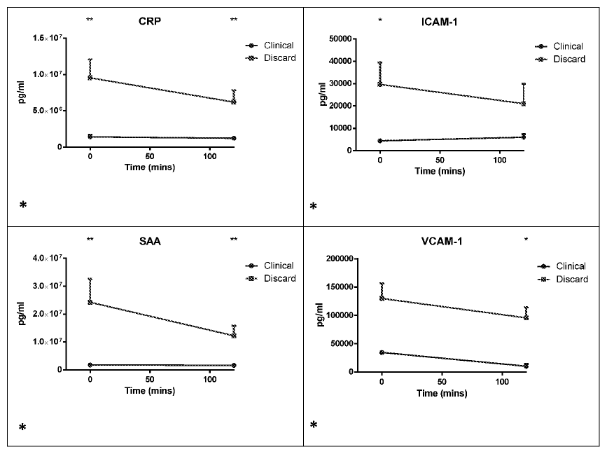

In certain embodiments, the method comprises detecting the concentration of IL-15 (e.g. human IL-15) in a perfusate fluid. IL-15 is a cytokine which binds to and signals through a complex of IL-2/IL-15 receptor beta chain and the common gamma chain. It is secreted by mononuclear phagocytes and dendritic cells and induces cell proliferation of NK cells. The canonical amino acid sequence of human IL-15 is disclosed under accession number UniProtKB P40933-1 (version 1). The amino acid sequence is shown in FIG. 6 (SEQ. ID. No. 4).

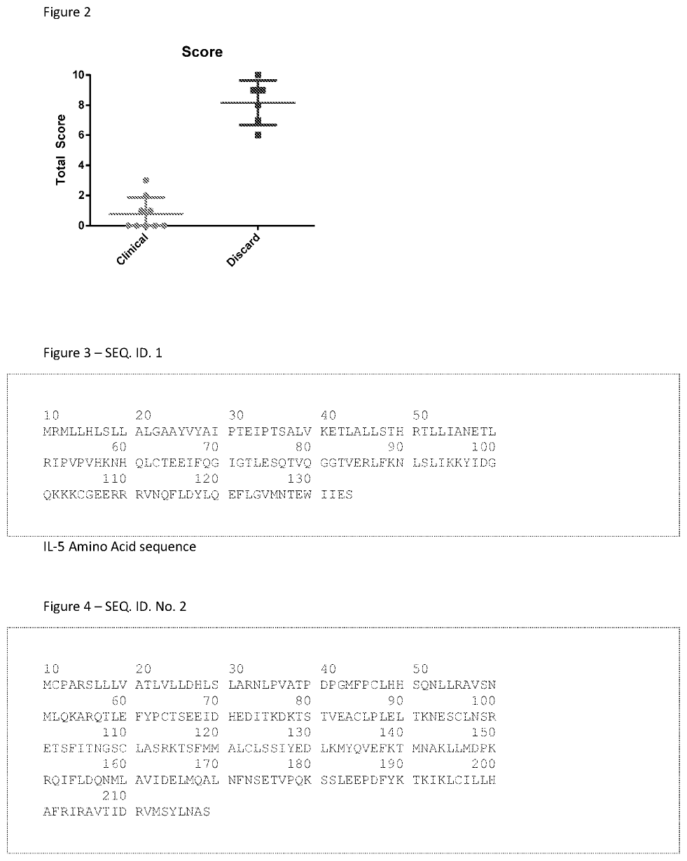

In certain embodiments, the method comprises detecting the concentration of C-reactive protein (CRP) (e.g. human CRP) in a perfusate fluid. CRP is an annular pentameric protein found in blood plasma. It is synthesized by the liver and activates the complement system by binding to phosphocholine expressed on the surface of dead or dying cells. The amino acid sequence of human CRP is disclosed under accession number UniProt P02741-1 (version 1). The amino acid sequence is shown in FIG. 7 (SEQ. ID. No. 5).

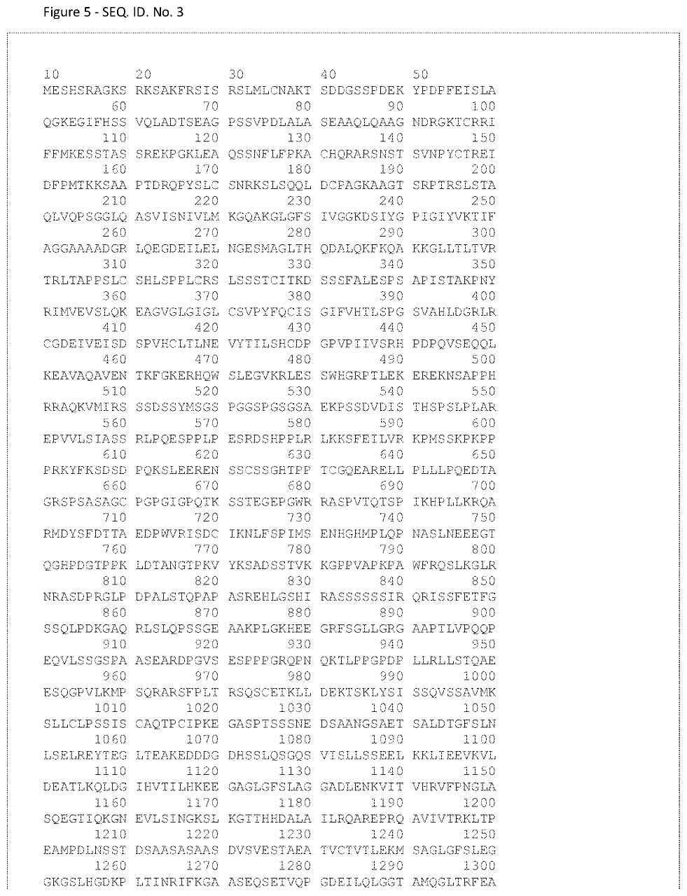

In certain embodiments, the method comprises detecting the concentration of VEGF in a perfusate fluid. VEGF (vascular endothelial growth factor) binds to VEGFR on a cell surface and plays a central role in angiogenesis, vasculogenesis and endothelial cell growth. There are a number of isoforms, the sequences of which are disclosed under Accession No. P15692-1 et al (version 2). The canonical sequence (VEGF206) is published under P15692-1. The sequence is shown in FIG. 8 (SEQ. ID. No. 6).

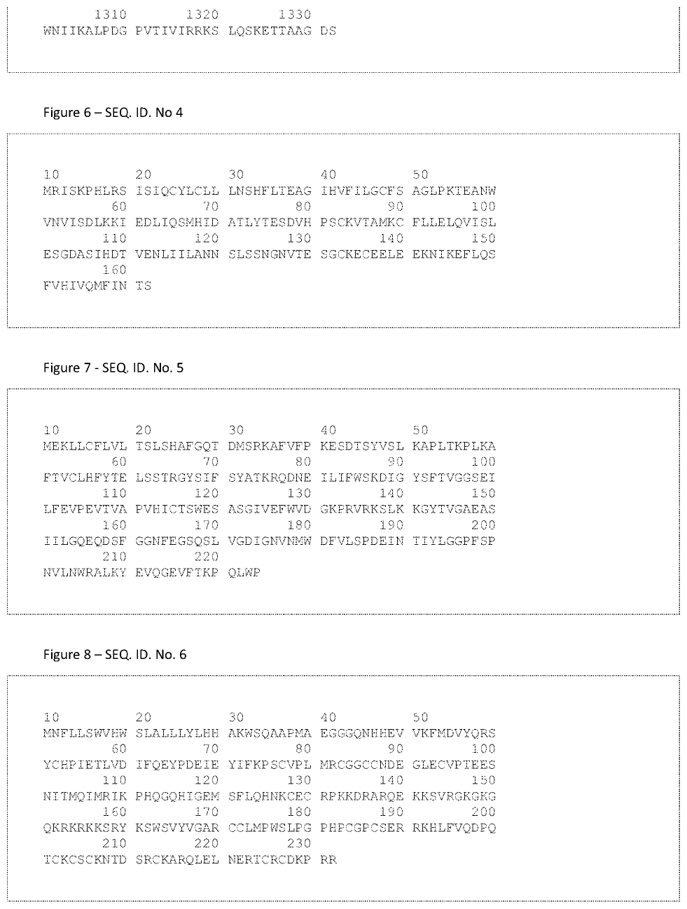

In certain embodiments, the method further comprises detecting the concentration of IL-7 (e.g. human IL-7) in a sample of perfusate fluid. IL-7 is a cytokine secreted by stromal cells in bone marrow and thymus. IL-7 is important for B and T cell development and is capable of stimulating the proliferation of lymphoid progenitors. The amino acid sequence of human IL-7 is disclosed under accession no. UniProtKB P13232 version 1 with the canonical sequence disclosed under P13231-1. The amino acid sequence of human IL-7 is shown in FIG. 9 (SEQ. ID. No. 7).

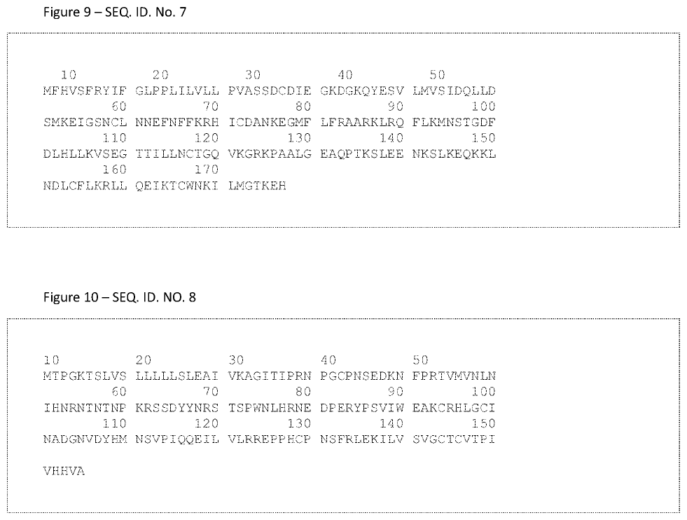

In certain embodiments, the method further comprises detecting the concentration of IL-17a (e.g. human IL-17a) in a sample of perfusate fluid. The amino acid sequence of human IL-17a is disclosed under accession number UniProtKB Q16552 version 1. A pro form of IL-17a is shown in FIG. 10 (SEQ. ID. No 8). The pro-form of the protein is subsequently cleaved to form mature IL-17a.

In certain embodiments, the predetermined value for the amount of IL-5 in the sample is between about 0.62 pg/ml and about 0.63 pg/ml, for example about 0.6221 pg/ml. In certain embodiments, the predetermined value for the amount of IL-12 in the sample is between about 14.4 pg/ml and 14.5 pg/ml. For example, the predetermined value for the amount of IL-12 is about 14.46 pg/ml.

In certain embodiments, the predetermined value for the amount of IL-15 in the sample is between about 2.19 pg/ml to about 2.20pg/ml. For example, the predetermined value for the amount of IL-15 may be about 2.192 pg/ml. In certain embodiments, the predetermined value for the amount of IL-16 is between about 1235 pg/ml and about 1250 pg/ml. For example, the predetermined value for the amount of IL-16 is about 1244 pg/ml.

In certain embodiments, the predetermined value for the amount of VEGF is between about 6.28 pg/ml and about 6.29 pg/ml. For example, the predetermined value of VEGF is about 6.285 pg/ml.

In certain embodiments, the predetermined value for the amount of CRP is between about 2.39 pg/ml and about 2.40 pg/ml. For example, the predetermined value for the amount of CRP may be about 2.394 pg/ml.

Detection Methods

Certain embodiments of the present invention comprise detecting the presence of one or more of the protein biomarkers described herein. In some embodiments, the sample of perfusate fluid may be processed prior to protein analysis for example by adding one or more components (such as detergents, buffers or salts). Aptly, the amount of protein biomarkers is quantified. The protein biomarkers may be detected by a variety of methods including for example, ELISA, Western Blot or radioimmunoassay. Immunohistochemical techniques can also be utilised for protein detection and quantification. In certain embodiments, the ELISA may be any known ELISA techniques including direct, indirect and/or sandwich ELISA.

In some embodiments, the proteins may be identified or confirmed using microarray techniques. In certain embodiments, the proteins are quantified by immunoassay. For example, a V-Plex immunoassay panel may be used. Aptly, the method comprises contacting the sample of perfusate fluid with one or more detection molecules. The detection molecule may comprise a capture antibody that is specific to one of the protein biomarkers described herein. In alternative embodiments, the detection molecule may comprise an aptamer.

In certain embodiments, a multiplex assay is used in which multiple capture antibodies are used, each capture antibody having a binding specificity for one of the protein biomarkers described herein. The assay may also involve the use of a labelled detection antibody which also binds to a protein biomarker described herein. Aptly, at least one of the capture antibody and labelled detection antibody is immobilised. In certain embodiments, the detection moiety is an electrochemiluminescence tag.

In certain embodiments, the method comprises contacting the sample of perfusate fluid with a capture antibody or fragment thereof which specifically binds to IL-5. In certain embodiments, the method comprises contacting the sample of perfusate fluid with a capture antibody or fragment thereof which specifically binds to IL-12. In certain embodiments, the method comprises contacting the sample of perfusate fluid with a capture antibody or fragment thereof which specifically binds to IL-15. In certain embodiments, the method comprises contacting the sample of perfusate fluid with a capture antibody or fragment thereof which specifically binds to IL-16. In certain embodiments, the method comprises contacting the sample of perfusate fluid with a capture antibody or fragment thereof which specifically binds to C-reactive protein (CRP). In certain embodiments, the method comprises contacting the sample of perfusate fluid with a capture antibody or fragment thereof which specifically binds to VEGF.

In certain embodiments, the method comprises contacting the sample with a plurality of capture antibodies to IL-5. In certain embodiments, the method comprises contacting the sample of perfusate fluid with a plurality of capture antibodies or fragments thereof which specifically bind to IL-12. In certain embodiments, the method comprises contacting the sample of perfusate fluid with a plurality of capture antibodies or fragments thereof which specifically bind to IL-15. In certain embodiments, the method comprises contacting the sample of perfusate fluid with a plurality of capture antibodies or fragments thereof which specifically bind to IL-16. In certain embodiments, the method comprises contacting the sample of perfusate fluid with a plurality of capture antibodies or fragments thereof which specifically bind to C-reactive protein (CRP). In certain embodiments, the method comprises contacting the sample of perfusate fluid with a plurality of capture antibodies or fragments thereof which specifically bind to VEGF.

In certain embodiments, the detection molecule comprises a binding moiety such as (1) a capture antibody or a universal anti-IgG antibody that is capable of binding to primary antibodies used as the ligand and (2) a detection moiety. In some embodiments, the binding moiety is a secondary antibody which binds specifically to the ligand.

In some embodiments, the ligand comprises a detection moiety (e.g. a fluorescent label). A detection moiety enables the direct or indirect detection and/or quantification of the complexes formed.

The antibodies used in the method e.g. the capture antibody may be any polyclonal antibodies, any monoclonal antibodies, including chimeric antibodies, humanized antibodies, bi-specific antibodies and domains and fragments of monoclonal antibodies including Fab, Fab′, F(ab′)2, scFv, dsFv, ds-scFv, dimers, minibodies, diabodies, and multimers thereof. Monoclonal antibodies can be fragmented using conventional techniques. Monoclonal antibodies may be from any animal origin, including birds and mammals (e.g., human, murine, donkey, sheep, rabbit, goat, guinea pig, camel, horse, or chicken), transgenic animals, or from recombinant sources. Monoclonal antibodies may be prepared using any methods known to those skilled in the art, including by recombination.

Typically, the antibody is isolated. An “isolated” antibody is an antibody that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In certain embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, silver stain.

An “antibody fragment” is a portion of an intact antibody that includes an antigen binding site of the intact antibody and thus retaining the ability to bind to the proteins listed herein. Antibody fragments include:
(i) Fab fragments, having V_{L}, C_{L}, V_{H} and CH1 domains;
(ii) Fab′ fragments, which is a Fab fragment having one or more cysteine residues at the C -terminus of the CH1 domain;
(iii) Fd fragments having V_{H} and CH1 domains;
(iv) Fd′ fragments having V_{H} and CH1 domains and one or more cysteine residues at the C-terminus of the CH1 domain;
(v) Fv fragments having the V_{L} and V_{H} domains of a single arm of an antibody;
(vi) dAb fragments (Ward et al., Nature 341, 544-546 (1989)) which consist of a VH domain;
(vii) isolated CDR regions;
(viii) F(ab′)2 fragments, a bivalent fragment including two Fab′ fragments linked by a disulphide bridge at the hinge region;
(ix) single chain antibody molecules (e.g. single chain Fv; scFv) (Bird et al, Science 242:423-426 (1988); and Huston et al., PNAS (USA) 85:5879-5883 (1988));
(x) “diabodies” with two antigen binding sites, comprising a heavy chain variable domain (VH ) connected to a light chain variable domain (VL) in the same polypeptide chain (see, e.g., EP 404,097 and WO 93/11161);
(xi) “linear antibodies” comprising a pair of tandem Fd segments (VH-CH1-VH-CH1) which, together with complementary light chain polypeptides, form a pair of antigen binding regions (Zapata et al. Protein Eng. 8 (10): 1057-1062 (1995); and U.S. Pat. No. 5,641,870).

Typically, the antibody is a recombinant monoclonal antibody. A “recombinant monoclonal antibody” is an antibody or antibody fragment produced using recombinant antibody coding genes.

Typically, the antibody is a monovalent Fab or bivalent Fab fragment. A “bivalent Fab fragment” may be considered as equivalent to a F(ab′)2 fragment and formed via dimerization. For example, a bivalent Fab fragment is formed via dimerization of a synthetic double helix loop helix motif (dHLX) or a bacterial alkaline phosphatase (AP) domain.

In certain embodiments, the antibody is a recombinant monoclonal antibody fragment converted into an immunoglobulin (Ig) format. For example, when an Fc region is required, the variable heavy and light chain genes may be cloned into vectors with the desired constant regions and co-transfected for expression in mammalian cells using methods known to those skilled in the art. In certain embodiments, antibody fragments are converted to human IgA, IgE, IgG1, IgG2, IgG3, IgG3 or IgM.

In certain embodiments, the method comprises detecting and/or quantifying the amount of protein in parallel. Thus, the method may comprise a multiplex assay step.

Scoring

In certain embodiments, the amount of the one or more protein biomarkers is analysed to determine whether the amount in the sample is above or below a predetermined cut-off value. The predetermined cut-off value is aptly established by performing Receiver Operator Characteristics (ROC) curve analysis. Sensitivity, specificity, positive predictive value (PPV) and negative predictive values (NPV) may also be calculated.

In certain embodiments, the method comprises assigning a score to the organ of 1 if the amount of protein biomarker is above the predetermined cut-off value. In certain embodiments, the method comprises assigning a score of 0 to the organ if the amount of protein biomarker in the sample is equal to below the predetermined cut-off value. In certain embodiments, the method further comprises combining the score assigned to one or more protein biomarkers. In certain embodiments, the method comprises determining that the organ is suitable for transplantation if the combined score is less than 3. In certain embodiments, the method further comprises determining that the organ is unsuitable for transplantation is the combined score is more than 3.

Methods of Surgery

In certain embodiments, the method comprises transplanting an organ which has been scored as suitable for transplantation into a subject.

The subject may be a human or non-human mammal. Aptly, the subject is human.

The organ may be selected from a kidney, a heart, a lung and a liver. In certain embodiments the organ is a liver.

In certain embodiments, the method further comprises transplanting an organ which is scored as suitable into a subject. In certain embodiments, the subject is suffering from liver cirrhosis. In certain embodiments, the subject is suffering from liver cirrhosis caused by non-alcoholic fatty liver disease (NAFLD). In certain embodiments, the subject is suffering from liver cancer.

### EXAMPLES

Materials and Methods

Hypothermic Oxygenated Perfusion

A preliminary series of 6 discarded livers were perfused to establish safety, followed by 10 clinical liver perfusions.

Hypothermic Oxygenated Perfusion (HOPE)

Discarded livers offered for research and suitable livers that will be transplanted were perfused in accordance with the following protocol.

Circuit Design

The circuit is manufactured to our design and specifications by Medtronic Ltd (Minneapolis, MN) with a heparin coating (Carmeda BioActive® surface). The feeding line splits to form hepatic artery and portal venous lines. The flows in the two limbs are measured independently, and pinch valves in the lines can control differential flow.

HOPE Protocol

All perfusions were carried out in a clean theatre perfusion room within the transplant theatre complex at the Freeman Hospital, Newcastle upon Tyne.

The protocol consists of dual arterial and portal-venous perfusion, using a fixed-pressure variable-flow algorithm. The arterial perfusion is non-pulsatile and a fully cannulated closed circuit was utilised.

The primary perfusate was Belzer University of Wisconsin solution with the addition of N-acetylcysteine, dexamethasone and antibiotics. The heater-cooler was set at 5° C., achieving a perfusate temperature of approximately 8° C. at the point of entry to the liver.
Equipment Required
   Metal trolley with BioMedicus Cardiopulmonary Bypass pump
   Attached to the trolley should be:
      Clamp for reservoir
      Pump
      Flow sensor x2
      Temperature probe x2
   Hirtz cooler
   Box containing sealed sterile circuit
   3×1 L bags of Belzer Machine Perfusion Solution
   Equipment box containing:
      Clamps
      Cannulae
      Pinch valves
      Syringes
      Drugs

Method:
1. Set up perfusion room
2. Drape a trolley for back table work as standard and drape the perfusion trolley also
3. Open extra plastic bowl onto perfusion trolley
4. Place pump and cooler on their respective trolleys adjacent to this
5. Plug everything in to wall sockets
6. Switch on cooler and set to 5° C.
7. Connect oxygen tubing to Medical Air theatre supply and lay tubing on the floor out of the way
8. Hand sterile clamps, syringes, and cannulae to scrubbed surgeon on back table
9. Open sterile circuit, surgeon to remove from sterile box and place on draped trolley
10. Perfusion surgeon to hand off reservoir and pump head through the back of the trolley (between drip stands), but retain the tubing holder, connectors, venous line (blue tip) and arterial lines (red tip x2)
11. Attach reservoir to clamp on metal pump trolley (must be as low as possible), ensuring no kinks in tubing
12. Attach pump head to magnetic pump
13. Attach flow sensors (small tube to blue sensor)
14. Connect air supply and open to 3 L/min
15. Attach pinch valves just distal to Y-split
16. Clamp reservoir outlet
17. Fill reservoir with 3L Belzer's Machine Perfusion Solution (MPS)
18. Clamp distal to pump head and release clamp proximal to it to prime and de-air pump head.
19. Clamp HA arterial line (just distal to Y-junction) and unclamp pump outlet to prime oxygenator/heat-exchanger
20. Switch on BioMedicus Console
21. Vented 3/8″ connector to connect sterile ends of PV and IVC lines
22. Start the pump at low rpm and use pump to prime venous side of circuit
23. Connect lines from cooler to the oxygenator and switch on the cooler and set to 5° C.
24. Connect temperature probe to connector on oxygenator outlet and reservoir inlet
25. Set up pressure lines (x2) ready to connect to cannulae and connect to P1 and P2 on back of console. P1 for HA, P2 for PV.
26. Draw up medications to add to circuit (NAC, dexamethasone, Benzyl Penicillin)

Open Liver from Transport Box
1. Perform back table dissection as standard
2. Cannulate suprahepatic IVC and tie in (purse-string with 2-0 Prolene)
3. Suture infrahepatic IVC
4. Cannulate portal vein (⅜″ tubing) and tie in (purse-string)
5. Cut off connector on end of PV cannula
6. Cannulate hepatic artery (¼″ tubing) and tie securely.
7. Prime cannulae with UW and clamp (no air)
8. Tie off any leaking branches
9. Stop the pump
10. Surgeon to clamp and separate the connector in the ⅜″ line leaving the connector on the PV side
11. Connect the cannulae (vented connector to PV, no connector end to IVC, ¼″ line to HA) using syringe to de-air
12. Connect pressure lines to HA and PV cannulae. Use syringe to fill pressure line and remove air. Open 3-way tap to air and zero (x2)
13. Zero flow sensors (x2)

Hypothermic Oxygenated Perfusion Procedure
1. Use pinch valve to clamp down on PV
2. Unclamp IVC line
3. Start the pump at 500 rpm
4. Unclamp HA line
5. Adjust rpm until HA pressure is 25 mmHg
6. Slowly open PV pinch valve
7. Adjust RPM and pinch valve to achieve PV pressure 2-3 mmHg and HA pressure 25 mmHg
8. The pinch valve may need to be readjusted to keep the differential pressures correct
9. It is important not to exceed these pressures as sheer stress will occur
10. Aim for total flow of 0.667 ml/kg/min but do not exceed pressures
11. Maintain perfusate temperate 8-10° c
12. Use infra-red thermometer to check liver surface temperature
13. Continue to pump, maintaining these parameters for 2 hours or until transplanting surgeon is ready to implant
14. Record temperature, pressure, flow and arterial and venous gases every 20 minutes.
15. Take samples for viability assessment at 20 minutes.

Removing from Pump
1. Turn pump dial to 0
2. Untie and remove IVC cannula first
3. Untie and remove other cannulae
4. Transfer liver in bowl to recipient for implantation

Storage
1. Discard of sterile tubing and reservoir
2. Wipe down all equipment
3. Restock and return equipment to perfusion lab

Equipment box contents
1. Line Clamps×6
2. Cannulae arterial and venous×2 each
3. Multiple blood gas syringes
4. Multiple insulin (1 ml) syringes
5. Pinch valves

Medications to Add to Circuit
N-acetylcysteine 600 mg
Dexamethasone 6.6 mg
Benzylpenicillin 600 mg

Sample Handling

Biopsies at 0, 60 and 120 minute time-points were obtained from discarded organs, and perfusate samples obtained at 20-minute intervals from all livers. Biopsies have been taken of the bile duct and portal vein at the end of perfusion. All biopsies were split into 3 for formalin, RNAlater and snap-frozen fresh. All perfusate and biopsy samples (excluding formalin) were stored at -80° C. Arterial and venous blood gases were performed at 20-minute intervals and recorded. Perfusion dynamics and temperature were also charted at 20-minute intervals.

The HOPE setup used is summarised in Table 1:

TABLE 1
hypothermic perfusion method

| | Newcastle |
|---|---|
| Type of Perfusion | D-HOPE |
| Arterial Perfusion | Non-pulsatile perfusion |
| IVC | Cannulated |
| Duration of Perfusion | 80-150 mins |
| Oxygenation | 20-30 kPa |
| Device | Medtronic |
| Temperature | 8-10° C. |
| Flow | Variable on pressure |
| Pressure | 3 mmHg (PV) |
| | 25 mmHg (HA) |
| Perfusate | Belzer UW |
| Additives | Antioxidant and vasodilator |
| Perfusate volume | 3 L |

Hypothermic Perfusion (n=16)

Hypothermic livers were divided into 2 groups — ones that were deemed transplantable on clinical grounds, or untransplantable on multiple factors. Livers that were deemed untransplantable were declined by all transplant centres in the UK, these are termed ‘discard’ livers (n=6). Clinical livers were classed as transplantable purely on clinical grounds, but were all high-risk livers that had been declined by at least one centre, and hence felt that ex-vivo perfusion could potentially provide benefit. Livers were allocated into groups sequentially, 6 discards were perfused followed by 10 clinical livers. All clinical livers were transplanted with good immediate clinical outcomes.

Samples of perfusate from all liver perfusions was collected at regular intervals and stored at −80° C. until the day of analysis.

The differences between the clinical and discard livers may not be exclusively due to differences in liver characteristics and performance as there was a significant difference in cold-ischaemic times (CIT). This discrepancy was unavoidable due to the logistics of the offering process. In clinical livers there are obvious time-pressures to transplant as soon as is feasible. However, discard livers undergo a full offering process to all centres before being offered for research, including sometimes awaiting biopsy results. Even after acceptance for research, the liver must then be transported prior to research taking place.

The introduction of clinical hypothermic perfusion has not affected the mean static cold storage time, but did increase the overall preservation time.

### Example 1

### Real-Time Pre-Implant Liver Assessment

Multiplex Analysis

Methods

Perfusate samples were taken at regular intervals from all livers perfused and stored at −80° C. for late analysis. A multiplex assay was performed on perfusate samples from all livers to assess for any biomarker that could differentiate between different groups of livers.

Perfusate samples and reagents were brought to room temperature. The following Mesoscale (Rockville Md., USA) multiplex plates were used:
V-PLEX Chemokine Panel 1
V-PLEX Pro-inflammatory Panel 1
V-PLEX Cytokine Panel 1
V-PLEX TH17 Panel 1
V-PLEX Angiogenesis Panel 1
V-PLEX Vascular Injury Panel 2

Calibration solutions were prepared in the appropriate diluent for each panel, with 4-fold serial dilutions. Perfusates were diluted 2-fold for time-0 time-points and by 10-fold for end time-points in the appropriate diluent for each plate. A combined detection antibody solution for each plate was prepared, by diluting each antibody 50-fold in the appropriate diluent for each plate. Plates were washed 3 times in PBS with 0.05% Tween 20 then 50 μL of diluted sample added to each well. Plates were incubated on a plate-shaker at room temperature for 2 hours. Plates were washed again 3 times then 25 μL of detection antibody solution was added to each well and incubated on a plate-shaker at room temperature for a further 2 hours. Plates were washed 3 times then 150 μL of read buffer added to each well and plate analysed on MESO QuickPlex SQ120 multiplex analyser.

In total 18 of the markers analysed showed a statistically significant difference in levels between the 2 groups in at least one time-point. Some of the readings were unobtainable as levels were outside the reference range.

The general trend in most markers analysed is that the levels in perfusate of discarded livers were often higher than clinical livers with some notable exceptions. Cytokines known to have anti-inflammatory actions such as IL-4, IL-10 and IL-13 seem to reverse this pattern, although of these only IL-13 was statistically significant at time-0.

The class of markers with most significant differences between the two groups were indicators of vascular injury such as serum amyloid A (SAA), vascular cell adhesion protein-1 (VCAM-1), intercellular adhesion molecule-1 (ICAM-1) and C-reactive protein (CRP). In all of these, a statistically significant difference was evident from the first time-point and continues to the end of perfusion.

Some angiogenesis markers such as vascular endothelial growth factor (VEGF), angiopoietin-1 receptor (Tyrosine kinase with immunoglobulin-like and EGF-like domains 2, TIE-2), and basic fibroblast growth factor (bFGF) were also statistically significant, although the end time-point of TIE-2 in discard livers was outside the reference range, most likely higher than range.

Tumour necrosis factor-β (TNF-β), important in the regulation of cell survival, was also significantly elevated in discarded livers. Granulocyte macrophage colony-stimulating factor (GM-CSF), part of the immune-inflammatory cascade was also significantly elevated in discarded livers.

A number of interleukins demonstrated a statistically significant difference between the 2 groups. IL-2, IL-7 and IL-15, all involved in regulating immune response, were significantly higher in discarded livers. Similarly, IL-5 which regulates eosinophil-mediated inflammation was also elevated.

IL-12, IL-16, IL-18 and IL-23 all have pro-inflammatory targets and were significantly elevated in discarded livers compared with clinical livers. IL23p40 also increases angiogenesis, in keeping with findings with angiogenic markers.

Analysis of Test Accuracy

In order for these markers to be of clinical use, they must be able to distinguish a clinical-grade liver from a discard liver with a high level of sensitivity and specificity. Receiver Operator Characteristics (ROC) curve analysis was performed to establish the optimum cut-off value and sensitivity, specificity, positive predictive value (PPV) and negative predictive values (NPV) were calculated, and the 10 tests with highest sensitivity and specificity were selected.

TABLE 2
Table 2 - Analysis of perfusate test accuracy

| | | Cut-off | | | | | | |
|---|---|---|---|---|---|---|---|---|
| Test | Timepoint | (pg/ml) | Sensitivity | Specificity | PPV | NPV | Area | p |
| IL5 | Start | 0.6221 | 100.00 | 88.89 | 85.71 | 100.00 | 0.963 | 0.003 |
| IL7 | End | 4.717 | 83.33 | 100.00 | 100.00 | 88.89 | 0.958 | 0.004 |
| IL12/23p70 | Start | 14.46 | 100.00 | 88.89 | 85.71 | 100.00 | 0.982 | 0.002 |
| IL15 | Start | 2.192 | 100.00 | 88.89 | 85.71 | 100.00 | 0.982 | 0.002 |
| IL16 | Start | 1244 | 83.33 | 88.89 | 83.33 | 88.89 | 0.926 | 0.006 |
| IL16 | End | 934.3 | 50.00 | 100.00 | 100.00 | 75.00 | 0.833 | 0.034 |
| IL17A | End | 3.646 | 100.00 | 100.00 | 100.00 | 100.00 | 1.00 | 0.004 |
| VEGF | Start | 6.285 | 83.33 | 77.78 | 71.43 | 87.50 | 0.833 | 0.034 |
| CRP | Start | 2.394 | 83.33 | 88.89 | 83.33 | 88.89 | 0.907 | 0.009 |
| CRP | End | 4.230 | 66.67 | 100.00 | 100.00 | 81.82 | 0.815 | 0.045 |

From this, 4 tests (IL5, ID 2, ID 5 and ID 7) had 100% sensitivity, that is in a discard liver, the value recorded was above the cut-off in all livers. 4 different tests also had 100% specificity that is in a clinical liver, the value recorded was below the cut-off in all cases. PPV represents the chances that the test is above the cut-off that this correctly identified a discarded liver, this was 100% in 4 tests. NPV is likely to be the most clinically relevant, this means that if the test is below the cut-off level, the chances of it being a clinical liver, this was also 100% in 4 tests. The only marker to have 100% sensitivity, specificity, PPV and NPV was IL17A with a cut-off value of 3.646 pg/ml.

In certain embodiments, combining the 10 tests above to produce a score would increase the accuracy in assessing the transplantability of a liver. As such a new scoring system was devised where 1 point was added for each test result that is above the established cut-off, giving a maximum score of 10. The total points for each liver perfused were added together and displayed below.

TABLE 3

| | Donor number | Total Score |
|---|---|---|
| Hypothermic Discard (6) | 116476 | 9 |
| | 117644 | 8 |
| | 118050 | 7 |
| | 118754 | 10 |
| | 118768 | 9 |
| | 119286 | 6 |
| Hypothermic Clinical (9) | 118802 | 3 |
| | 118856 | 2 |
| | 120127 | 0 |
| | 120143 | 0 |
| | 121791 | 0 |
| | 122173 | 1 |
| | 123013 | 0 |
| | 126051 | 1 |
| | 126380 | 0 |

There is a statistically significant difference in the scores of the clinical and discard livers (t-test, p<0.0001). None of the clinical livers had a score over 3, and none of the discard livers had a score under 6.

Alternatively, in certain embodiments, and to improve the clinical applicability of the scoring system, tests that are reliable at the start of perfusion were used so that a decision on transplantability could be made without having to wait until the end of perfusion. To do this, a scoring system was applied using only the 6 tests above that were taken at the start of perfusion. The maximum score was then 6. The results are displayed below.

TABLE 4

| | Donor number | Total Score |
|---|---|---|
| Hypothermic Discard (6) | 116476 | 6 |
| | 117644 | 5 |
| | 118050 | 6 |
| | 118754 | 6 |
| | 118768 | 6 |
| | 119286 | 4 |
| Hypothermic Clinical (9) | 118802 | 3 |
| | 118856 | 2 |
| | 120127 | 0 |
| | 120143 | 0 |
| | 121791 | 0 |
| | 122173 | 1 |
| | 123013 | 0 |
| | 126051 | 1 |
| | 126380 | 0 |

Using the start-point scoring system, there is less differentiation between groups, with the lowest scoring discard liver having a score of 4 versus the highest-scoring clinical liver score of 3. There is still a clear statistically significant difference between the two groups (t-test, p=<0.0001). In certain embodiments, the method may comprise testing the 6 start-point tests and if this produces a score>2, to proceed to complete the full 10-point scoring system later in the perfusion.

### SUMMARY

Being able to use perfusate samples to aid in the decision on whether to transplant a liver or not has the potential to increase the numbers of livers available for transplant as well as potentially improving the accuracy of that decision with more objective and repeatable measures. A number of potential markers have been analysed.

The analysis of hypothermic livers compared two groups. Livers were essentially allocated to groups by clinical decision-making as discard livers had been declined by all centres purely on data available on donor factors and the retrieval process including visual inspection of the liver by the surgeon.

A number of biomarkers have been identified that differentiate between a liver that has been clinically deemed transplantable (hypothermic clinical livers) and livers that have been declined by all centres. These include vascular injury markers and angiogenesis markers. Pro-inflammatory cytokines were significantly elevated in discard livers and conversely, anti-inflammatory cytokines tended to show the opposite effect. In addition, cytokines involved in regulating the immune response were also significantly elevated in the discard group. One or more of these in combination could aid in the decision-making process on the transplantability of a liver. Two compound scores using biomarkers have been proposed, both clearly distinguishing between clinical and discard livers. The 6-point scoring system has the advantage of only using perfusate taken at the start of perfusion, so would allow for an early decision on transplantability. A potential route to clinical application could therefore be to run the 6-point test at the start of perfusion, and if a more equivocal result (e.g. >2) was obtained, perfusion could be continued and a further completion 10-point test carried out at the end of perfusion before a decision on transplantability is made.

### CONCLUSION

Aptly in certain embodiments the test focuses on 6 specific proteins (Interleukin 5, IL-12, IL-15, IL-16, CRP and VEGF) found in the circulating perfusate at 20 minutes after the initiation of organ perfusion. This biomarker has the potential to be an objective test for liver transplant viability. High scores of these compounds beyond the cut-off would mean that a liver was not viable for transplantation. All the livers scoring 3 or less were successfully transplanted with no clinically significant ischaemic cholangiopathy.

Throughout the description and claims of this specification, the words “comprise” and “contain” and variations of them mean “including but not limited to” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any foregoing embodiments. The invention extends to any novel one, or novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A method of assessing suitability of an explant organ for transplantation into a subject in need thereof, the method comprising:
a) obtaining a sample of a perfusate fluid in which an explant organ is located;
b) determining an amount in the sample of at least three proteins selected from:
i) IL-5;
ii) IL-12;
iii) IL-15;
iv) IL-16;
v) C-reactive protein (CRP); and
vi) VEGF; and
c) determining whether the explant organ is suitable for transplantation based on whether a respective amount of each protein in the sample is greater or less than a respective predetermined value.

2. The method according to claim 1, which comprises determining that the explant organ is suitable for transplantation if the respective amount of each of the at least three proteins is less than the respective predetermined value.

3. The method according to claim 1, wherein the predetermined value(s) are selected from one or more of the group consisting of:
a) between about 0.62 pg/ml to about 0.63 pg/ml of IL 5;
b) between about 14.4 pg/ml to about 14.5 pg/ml of IL 12;
c) between about 2.19 pg/ml to about 2.2 pg/ml of IL 15;
d) between about 1235 pg/ml to about1250 pg/ml of IL 16;
e) between about 6.28 pg/ml to about 6.29 pg/ml of VEGF; and
e) between about 2.39 pg/ml to about 2.4 pg/ml CRP.

4. The method according to claim 1, wherein step (b) of determining an amount in the sample comprises quantifying the amount present in the sample of at least three proteins selected from:
i) IL-5;
ii) IL-12;
iii) IL-15;
iv) IL-16;
v) CRP; and
vi) VEGF; and
wherein the step of determining whether the explant organ is suitable for transplantation comprises;
a) for each protein, determining whether the amount of the protein is greater or less than a predetermined value;
b) assigning a score of 1 to the protein if the amount is greater than the predetermined value; and
c) adding together the score of each protein; wherein the explant organ is determined as suitable for transplantation if the additive score of the proteins is less than 3.

5. The method according to claim 1, wherein step (b) of determining an amount in the sample comprises quantifying the amount present in the sample of at least four proteins selected from:
i) IL-5;
ii) IL-12;
iii) IL-15;
iv) IL-16;
v) CRP; and
vi) VEGF, and
wherein the step of determining whether the explant organ is suitable for transplantation comprises;
a) for each protein, determining whether the amount of the protein is greater or less than a predetermined value;
b) assigning a score of 1 to the protein if the amount is greater than the predetermined value; and
c) adding together the score of each protein; wherein the explant organ is determined as suitable for transplantation if the additive score of the proteins is less than 3.

6. The method according to claim 1, wherein step (b) of determining an amount in the sample comprises quantifying the amount present in the sample of at least five proteins selected from:
i) IL-5;
ii) IL-12;
iii) IL-15;
iv) IL-16;
v) CRP; and
vi) VEGF, and
wherein the step of determining whether the explant organ is suitable for transplantation comprises;
a) for each protein, determining whether the amount of the protein is greater or less than a predetermined value;
b) assigning a score of 1 to the protein if the amount is greater than the predetermined value; and
c) adding together the score of each protein; wherein the explant organ is determined as suitable for transplantation if the additive score of the proteins is less than 3.

7. The method according to claim 1, which comprises:
quantifying the amount present in the sample of IL-5, IL-12, IL-15; IL-16; CRP; and VEGF;
assigning a score of 1 to the protein if the amount is greater than the predetermined value; and
adding together the score of each protein; wherein the explant organ is determined as suitable for transplantation if the additive score of the proteins is less than 3.

8. The method according to claim 1, wherein:
the explant organ is undergoing hypothermic perfusion;
(ii) the method further comprises, prior to step (a), locating the explant organ in the perfusate fluid;
(iii) the method further comprises inserting an organ into a perfusion machine comprising the perfusate fluid;
(iv) the perfusate fluid has a temperature of approximately 5 -10° C.;
(v) the explant organ is undergoing hypothermic perfusion; and/or
(vi) the sample is obtained approximately 15-30 minutes subsequent to the initiation of the perfusion.

9-13. (canceled)

14. The method according to claim 1, wherein:
(a) the perfusate fluid comprises one or more of the following components:
i) pentafraction;
ii) lactobionic acid;
iii) potassium phosphate monobasic;
iv) magnesium sulfate heptahydrate;
v) raffinose pentahydrate;
vi) adenosine;
vii) allopurinol;
viii) glutathione;
ix) potassium hydroxide; and
x) sodium hydroxide:
(b) the perfusate fluid further comprises N-acetylcysteine, dexamethasone and benzylpenicillin; and/or
(c) the organ is a liver.

15-16. (canceled)

17. The method according to claim 1, which further comprises determining the amount in the sample of at least one protein selected from:
a) IL-7;
b) IL-17a;
c) IL-16; and
d) CRP,
wherein the sample is obtained between about 80 minutes and 150 minutes after the initiation of perfusion and further wherein the method comprises;
i) determining whether the amount of the at least one protein is greater or less than a predetermined value;
ii) assigning a score of 1 to the protein if the amount is greater than the predetermined value; and
ii) adding together the score of each protein; wherein the explant organ is determined as suitable for transplantation if the additive score of the proteins is less than 3.

18. The method according to claim 17, which comprises, if the amount of more than one protein is determined, determining the amounts of proteins simultaneously.

19. The method according to claim 1, wherein the step of detecting the amount of the at least one protein comprises performing an immunoassay, wherein optionally the immunoassay is selected from an ELISA, a radioimmunoassay, automated immunoassay, cytometric based assay and immunoprecipitation assay.

20. The method according to any preceding claim claim 1, which further comprises contacting the sample with at least one detection molecule, wherein the detection molecule is specific to a protein selected from:
i) IL-5;
ii) IL-12;
iii) IL-15;
iv) IL-16;
v) C-reactive protein (CRP); and
vi) VEGF.

21. The method according to claim 20, which comprises contacting the sample with a plurality of detection molecules, the plurality of detection molecules comprising a detection molecule specific to a protein selected from:
i) IL-5;
ii) IL-12;
iii) IL-15;
iv) IL-16;
v) C-reactive protein (CRP); and
vi) VEGF.

22. The method according to claim 21, wherein the detection molecule comprises an antibody or fragment thereof.

23. The method according to claim 1, wherein:
(i) the method is an ex vivo method; and/or
(ii) the organ is obtained from a donor after death.

24. (canceled)

25. A method of transplanting an organ into a subject recipient comprising:
a) performing the method of claim 1; and
b) transplanting the organ into the subject recipient if the organ is assigned a score of 3 or less; or discarding the organ if the organ is assigned a score of 4 or greater.

26. The method according to claim 25, wherein:
(i) the organ is a liver, e.g. a human liver;
(ii) the method further comprises obtaining the organ from a donor who has undergone circulatory death prior to donation;
(iii) the method further comprises placing the organ in a hypothermic perfusion system; and/or
(iv) the subject is a human.

27-29. (canceled)

30. An assay device for use in determining whether an organ is suitable for transplantation into a subject, the assay comprising:
a) at least one reaction chamber; and
b) a plurality of detection molecules, the plurality comprising detection molecules that are specific to a protein selected from:
i) IL-5;
ii) IL-12;
iii) IL-15;
iv) IL-16;
v) C-reactive protein (CRP); or
vi) VEGF.

31. The assay device according to claim 30, wherein:
(i) the assay further comprises a plurality of detection molecules, the plurality comprising detection molecules which are specific to:
a) IL-7, or
b) IL-17a;
(ii) the detection molecules are antibodies or fragments thereof; and/or
(iii) the detection molecules are labelled.

32-33. (canceled)


SEQUENCE LISTING (8 sequences)

SEQ ID NO: 1 - protein, 134 aa, Homo sapiens
MRMLLHLSLLALGAAYVYAIPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 2 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 3 - protein, 1332 aa, Homo sapiens
MESHSRAGKSRKSAKFRSISRSLMLCNAKTSDDGSSPDEKYPDPFEISLAQGKEGIFHSSVQLADTSEAGPSSVPDLALASEAAQLQAAGNDRGKTCRRIFFMKESSTASSREKPGKLEAQSSNFLFPKACHQRARSNSTSVNPYCTREIDFPMTKKSAAPTDRQPYSLCSNRKSLSQQLDCPAGKAAGTSRPTRSLSTAQLVQPSGGLQASVISNIVLMKGQAKGLGFSIVGGKDSIYGPIGIYVKTIFAGGAAAADGRLQEGDEILELNGESMAGLTHQDALQKFKQAKKGLLTLTVRTRLTAPPSLCSHLSPPLCRSLSSSTCITKDSSSFALESPSAPISTAKPNYRIMVEVSLQKEAGVGLGIGLCSVPYFQCISGIFVHTLSPGSVAHLDGRLRCGDEIVEISDSPVHCLTLNEVYTILSHCDPGPVPIIVSRHPDPQVSEQQLKEAVAQAVENTKFGKERHQWSLEGVKRLESSWHGRPTLEKEREKNSAPPHRRAQKVMIRSSSDSSYMSGSPGGSPGSGSAEKPSSDVDISTHSPSLPLAREPVVLSIASSRLPQESPPLPESRDSHPPLRLKKSFEILVRKPMSSKPKPPPRKYFKSDSDPQKSLEERENSSCSSGHTPPTCGQEARELLPLLLPQEDTAGRSPSASAGCPGPGIGPQTKSSTEGEPGWRRASPVTQTSPIKHPLLKRQARMDYSFDTTAEDPWVRISDCIKNLFSPIMSENHGHMPLQPNASLNEEEGTQGHPDGTPPKLDTANGTPKVYKSADSSTVKKGPPVAPKPAWFRQSLKGLRNRASDPRGLPDPALSTQPAPASREHLGSHIRASSSSSSIRQRISSFETFGSSQLPDKGAQRLSLQPSSGEAAKPLGKHEEGRFSGLLGRGAAPTLVPQQPEQVLSSGSPAASEARDPGVSESPPPGRQPNQKTLPPGPDPLLRLLSTQAEESQGPVLKMPSQRARSFPLTRSQSCETKLLDEKTSKLYSISSQVSSAVMKSLLCLPSSISCAQTPCIPKEGASPTSSSNEDSAANGSAETSALDTGFSLNLSELREYTEGLTEAKEDDDGDHSSLQSGQSVISLLSSEELKKLIEEVKVLDEATLKQLDGIHVTILHKEEGAGLGFSLAGGADLENKVITVHRVFPNGLASQEGTIQKGNEVLSINGKSLKGTTHHDALAILRQAREPRQAVIVTRKLTPEAMPDLNSSTDSAASASAASDVSVESTAEATVCTVTLEKMSAGLGFSLEGGKGSLHGDKPLTINRIFKGAASEQSETVQPGDEILQLGGTAMQGLTRFEAWNIIKALPDGPVTIVIRRKSLQSKETTAAGDS

SEQ ID NO: 4 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 5 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 6 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 7 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 8 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA